(19) United States
(12) Patent Application Publication
(10) Pub. No.: US 2019/0204268 A1
(43) Pub. Date: Jul. 4, 2019

(54) NON-ENZYMATIC SENSOR

(71) Applicant: CASE WESTERN RESERVE UNIVERSITY, Cleveland, OH (US)

(72) Inventors: Chung Chiun Liu, Cleveland Heights, OH (US); Yifan Dai, Cleveland Heights, OH (US)

(21) Appl. No.: 16/238,303

(22) Filed: Jan. 2, 2019

Related U.S. Application Data
(60) Provisional application No. 62/612,938, filed on Jan. 2, 2018.

(51) Int. Cl. G01N 27/49; A61B 5/145; G01N 27/327; G01N 27/407; G01N 27/416; G01N 27/447

(52) U.S. Cl. CPC G01N 27/49; A61B 5/14532; G01N 27/3272; G01N 27/4473; G01N 27/4076; G01N 27/4166; G01N 27/3278

(57) ABSTRACT

A non-enzymatic sensor for the detection of glucose includes a substrate, a working electrode formed on a surface of the substrate; a counter electrode formed on the surface of the substrate; a dielectric layer covering a portion of the working electrode and counter electrode and defining an aperture exposing other portions of the working electrode and counter electrode; and a cuprous oxide (Cu₂O) film electrodeposited on the working electrode.

### RELATED APPLICATION

This application claims priority from U.S. Provisional Application No. 62/612,938, filed Jan. 2, 2018, the subject matter of which is incorporated herein by reference in its entirety.

### BACKGROUND

An abnormal glucose concentration level is directly related to diabetes, obesity, hyperglycemia and encephalopathy. Therefore, a cost-effective, accurate, consistent glucose sensor is important in medical diagnosis. Measuring glucose concentration is also important for bio-processing and bio-reactor applications, as well as for home care usage. Current glucose sensors are typically based on an enzymatic mechanism with the advantages of low cost and simple operation. However, an enzyme-based biosensor is limited in accuracy and the reproducibility of measurements is only fair due to the loss of enzyme activity over time. Furthermore, the enzyme also has limited active life time affecting the manufacturing and shelf-life of the glucose sensor. Hence, a cost-effective, highly-accuracy, highly-reproducible, non-enzymatic glucose sensor is desirable for medical applications and bio-processing involving glucose as a reactant or product.

### SUMMARY

Embodiments described herein relate to a non-enzymatic sensor for detecting, identifying, quantifying, and/or determining the amount, concentration, or level of glucose in a bodily sample, and particularly relates to a non-enzymatic sensor for detecting, identifying, quantifying, and/or determining the amount, concentration, or level of glucose in a biological or bodily sample, such as breath, blood, and other physiological fluids.

The non-enzymatic sensor includes a substrate, a working electrode formed on a surface of the substrate, a counter electrode formed on the surface of the substrate, a dielectric layer covering a portion of the working electrode and counter electrode and defining an aperture exposing other portions of the working electrode and counter electrode.

The non-enzymatic sensor also includes a cuprous oxide (Cu₂O) film that is electrodeposited on the working electrode. Electrodeposition of cuprous oxide (Cu₂O) film can be accomplished in a relatively short deposition time and be substantially reproducible.

The non-enzymatic sensor can be used for the detection of glucose in ionic solution, blood serum and other test media. The non-enzymatic sensor showed a linear response toward glucose. This shows that the non-enzymatic glucose sensor is not only suitable for biomedical single-use in vitro application but also for long-term glucose monitoring in industrial processes.

### BRIEF DESCRIPTION OF THE DRAWINGS

FIG. 1 is a schematic illustration of a biosensor in accordance with an aspect of the application.

FIG. 2 illustrates a comparison of gold sensor prototype and prepared cuprous oxide film sensor.

FIG. 3(A) illustrates SEM nanoparticle structure of cuprous oxide film.

FIG. 3(B) illustrates images showing TOF-SIMS evaluation of the homogeneity of the sensor's surface.

FIG. 3(C) illustrates a plot showing the depth profile for the thickness of the cuprous oxide film.

FIG. 4 illustrates high resolution XPS spectrums of copper photoelectron peaks compared on two different samples. The take-off angle on one of the samples verified the consistency of the measurement at different thicknesses.

FIG. 5(A) illustrates DPV measurements of glucose concentrations ranging from 200 mg/dL to 50 mg/dL.

FIG. 5(B) illustrates calibration linear relationship of DPV current outputs and concentrations of glucose.

FIG. 5(C) illustrates the detection limit of the cuprous oxide sensor by using DPV was 0.2 mg/dl.

FIG. 6 illustrates chronoamperometry measurement of glucose concentrations ranging from 200 mg/dL to 50 mg/dL.

FIG. 7 illustrates single-potential amperometric voltammetry measurement of glucose concentrations ranging between 50 mg/dL and 200 mg/dL.

FIG. 8A illustrates differential pulse voltammetry measurement of glucose concentrations ranging from 50 mg/dL to 200 mg/dL in undulated human serum.

FIG. 8B illustrates calibration linear relationship of DPV current outputs and concentrations of glucose.

FIG. 9 illustrates interference tests from uric acid and ascorbic acid performed by DPV.

FIGS. 10(A-D) illustrate the detection response based on different metal films (A) copper, (B) nickel, (C) platinum, (D) cuprous oxide.

### DETAILED DESCRIPTION

Unless specifically addressed herein, all terms used have the same meaning as would be understood by those of skilled in the art of the subject matter of the application. The following definitions will provide clarity with respect to the terms used in the specification and claims.

The term “monitoring” refers to the use of results generated from datasets to provide useful information about an individual or an individual's health or disease status. “Monitoring” can include, for example, determination of prognosis, risk-stratification, selection of drug therapy, assessment of ongoing drug therapy, determination of effectiveness of treatment, prediction of outcomes, determination of response to therapy, diagnosis of a disease or disease complication, following of progression of a disease or providing any information relating to a patient's health status over time, selecting patients most likely to benefit from experimental therapies with known molecular mechanisms of action, selecting patients most likely to benefit from approved drugs with known molecular mechanisms where that mechanism may be important in a small subset of a disease for which the medication may not have a label, screening a patient population to help decide on a more invasive/expensive test, for example, a cascade of tests from a non-invasive blood test to a more invasive option such as biopsy, or testing to assess side effects of drugs used to treat another indication.

The term “quantitative data” or “quantitative level” or “quantitative amount” refers to data, levels, or amounts associated with any dataset components (e.g., markers, clinical indicia,) that can be assigned a numerical value.

The term “subject” refers to a human or another mammal. Typically, the terms “subject” and “patient” are used herein interchangeably in reference to a human individual.

The term “bodily sample” refers to a sample that may be obtained from a subject (e.g., a human) or from components (e.g., tissues) of a subject. The sample may be of any biological tissue or fluid with, which glucose may be assayed. Frequently, the sample will be a “clinical sample”, i.e., a sample derived from a patient. Such samples include, but are not limited to, bodily fluids, e.g., saliva, breath, urine, blood, plasma, or sera; and archival samples with known diagnosis, treatment and/or outcome history. The term biological sample also encompasses any material derived by processing the bodily sample. Processing of the bodily sample may involve one or more of, filtration, distillation, extraction, concentration, inactivation of interfering components, addition of reagents, and the like.

The terms “control” or “control sample” refer to one or more biological samples isolated from an individual or group of individuals that are normal (i.e., healthy). The term “control”, “control value” or “control sample” can also refer to the compilation of data derived from samples of one or more individuals classified as normal.

The terms “normal” and “healthy” are used interchangeably. They can, for example, refer to an individual or group of individuals who have not shown any symptoms of diabetes, and have not been diagnosed with diabetes. In certain embodiments, normal individuals have similar sex, age, body mass index as compared with the individual from which the sample to be tested was obtained. The term “normal” is also used herein to qualify a sample isolated from a healthy individual.

The terms “control” or “control sample” refer to one or more biological samples isolated from an individual or group of individuals that are normal (i.e., healthy). The term “control”, “control value” or “control sample” can also refer to the compilation of data derived from samples of one or more individuals classified as normal, and/or one or more individuals diagnosed with diabetes.

Embodiments described herein relate to a non-enzymatic sensor for detecting, identifying, quantifying, and/or determining the amount, concentration, or level of glucose in a bodily sample, and particularly relates to a non-enzymatic sensor for detecting, identifying, quantifying, and/or determining the amount, concentration, or level of glucose in a biological or bodily sample, such as breath, blood, and other physiological fluids.

Abnormal glucose concentration level is directly related to diabetes, obesity, hyperglycemia and encephalopathy. Therefore, cost-effective, accurate, consistent glucose sensor is important in medical diagnosis. Measuring glucose concentration is also important for bio-processing and bio-reactor applications as well as for home care usage. Current glucose sensors are mostly based on enzymatic mechanism with the advantages in low cost and simple operation. However, enzyme-based biosensor has the disadvantages in accuracy and reproducibility due to the activity loss of enzyme over time. Furthermore, enzymes have a limited active life time affecting the production and shelf-life of an enzyme-based glucose sensor. Hence, a cost-effective, high-accuracy, high-reproducibility, non-enzymatic glucose sensor is desirable for medical applications and bio-processing involving glucose as a reactant or product.

In some embodiments, the non-enzymatic sensor described herein includes a substrate, a working electrode formed on a surface of the substrate, a counter electrode formed on the surface of the substrate, a dielectric layer covering a portion of the working electrode and counter electrode and defining an aperture exposing other portions of the working electrode and counter electrode.

The non-enzymatic sensor also includes a cuprous oxide (Cu₂O) film that is electrodeposited on the working electrode. Electrodeposition of cuprous oxide (Cu₂O) film can be accomplished in a relatively short deposition time and be substantially reproducible.

As shown in the following reaction, the Cu⁺ ion can be reduced to Cu₂O by D-glucose in an alkaline solution having following the equilibrium:

2Cu⁺+C₆H₁₂O₆+4OH⁻→Cu₂O+C₆H₁₂O₇+2H₂O

The oxidation of glucose can be detected electrochemically using the sensor to allow quantification or determination of the amount, concentration, or level of glucose in a sample.

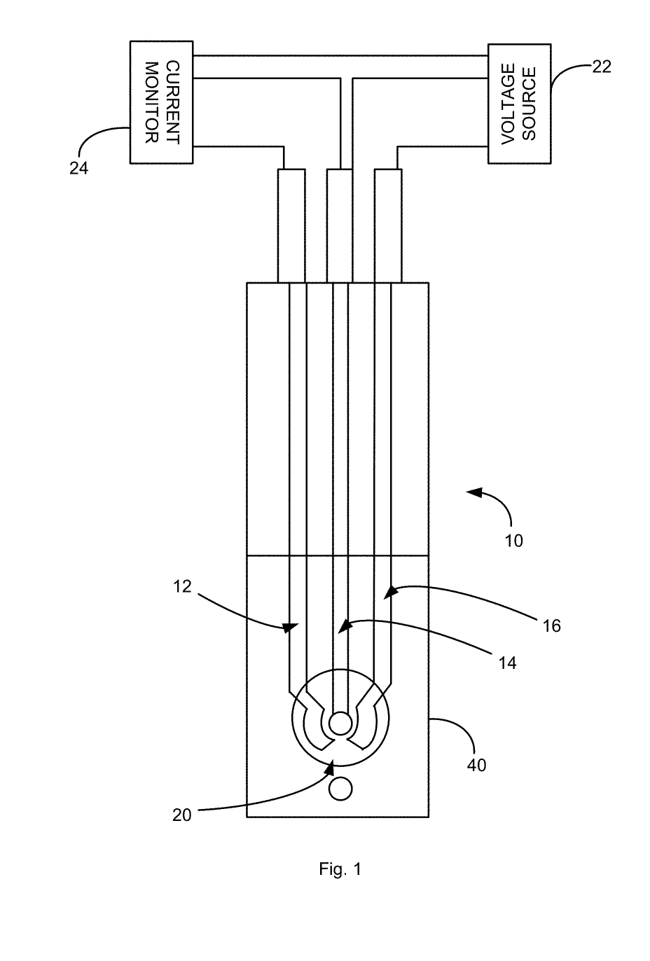

FIG. 1 illustrates a sensor 10 in accordance with an embodiment of the application. The sensor 10 is a three-electrode sensor including a counter electrode 12, a working electrode 14, and a reference electrode 16 that are formed on the surface of a substrate. A dielectric layer 40 covers a portion of the working electrode 12, counter electrode 14 and reference electrode 16. The dielectric layer 40 includes an aperture 20 which define a detection region of the working electrode 12, counter electrode 14, reference electrode 16 that is exposed to samples in which the concentration of glucose is detected.

The working electrode 14 include a metalized film with working surface and a layer or film of cuprous oxide (Cu₂O) film that is electrodeposited on the working electrode.

A voltage source 22 is connected to the working and reference electrodes 14, 16. A current measuring device 24 is connected to the working and counter electrodes 14, 12 to measure the current when a sample containing glucose contacts the detection region 20 of the sensor 10.

The non-enzymatic glucose sensor can be made using a thin film, thick film, and/or ink-jet printing technique, especially for the deposition of multiple electrodes on a substrate. The thin film process can include physical or chemical vapor deposition. Electrochemical sensors and thick film techniques for their fabrication are discussed in U.S. Pat. No. 4,571,292 to C. C. Liu et al., U.S. Pat. No. 4,655,880 to C. C. Liu, and co-pending application U.S. Ser. No. 09/466,865, which are incorporated by reference in their entirety.

In some embodiments, the working electrode, counter electrode, and reference electrode may be formed using laser ablation, a process which can produce elements with features that are less than one-thousandth of an inch. Laser ablation enables the precise definition of the working electrode, counter electrode, and reference electrode as well as electrical connecting leads and other features, which is required to reduce coefficient of variation and provide accurate measurements. Metalized films, such as Au, Pd, and Pt or any metal having similar electrochemical properties, that can be sputtered or coated on plastic substrates, such as PET or polycarbonate, or other dielectric material, can be irradiated using laser ablation to provide these features.

In one example, a gold film with a thickness of about 300 to about 2000 A can be deposited by a sputtering technique resulting in very uniform layer that can be laser ablated to form the working and counter electrodes. The counter electrode can use other materials. However, for the simplicity of fabrication, using identical material for both working and counter electrodes will simplify the fabrication process providing the feasibility of producing both electrodes in a single processing step. An Ag/AgCl reference electrode, the insulation layer, and the electrical connecting parts can then be printed using thick-film screen printing technique.

In some embodiments, the overall dimensions of an individual sensors are chosen to be 33.0×8.0 mm². The total width of each individual biosensor is approximately 2.8 mm with a working electrode of 1.0 mm in diameter sufficiently to accommodate up to a 5 μL sample volume. These sizes can be changed as needed.

In some embodiments, a three-electrode base electrochemical sensor can be formed where both working and counter electrodes are thin gold films of about 10 nm in thickness. Other metals can also be used for the fabrication of the working and the counter electrodes. The thin gold film can be deposited using roll-to-roll sputtering technique. Hence, the production of the gold-film based sensor is very cost effective and the gold electrode elements are very uniform and reproducible which are very practical and unique for single-use, in situ applications. Any other similar technique in deposition of any other metals can be used.

The reference electrode can be a thick-film printed Ag/AgCl electrode. Other types of the reference electrode and the formation method of the reference electrode can also be employed. In one example, the overall dimensions of an individual sensor were 33.0×8.0 mm². The working electrode area was 1.54 mm² accommodating 10-15 μL of liquid test sample. The dimensions and configuration of this sensor can be varied. The employment of known micro-fabrication processes, such as sputtering physical vapor deposition, laser ablation and thick film printing techniques resulting in producing a high-reproducible and low-cost single-use disposable biosensors.

A 3-step pretreatment procedure can be applied to the sensor in order to eliminate any naturally formed oxide of the gold or other metal surface resulting in a significant increase in the electrode charge transfer ability and enhancement in the reproducibility of the sensor. Typically, a batch of 8 sensors can be immersed in a 2M KOH solution for 15 min. Any other number of the sensors used in a batch is an option. After rinsing with copious amount of deionized (DI) water, the sensors are placed in 0.05 M concentrated H₂SO₄ solution (95.0 to 98.0 w/w %) for another 15 min DI water is then used to rinse the sensor prototypes. The sensors are then placed in a 0.05 M concentrated HNO₃ solution (70% w/w %) for another 15 minutes. The sensors are rinsed once more time with DI water and are dried in a steam of nitrogen. Other concentration of the KOH, H₂SO₄ and HNO₃ can also be used. The selected concentrations of the chemicals used in this chemical pretreatment step is to maintain the integrity of the thin gold film based sensor and retaining the clean surface of the electrode elements. Other means to accomplish this objective can also be sued, including using ethanol and DI water cleaning procedure.

The cuprous oxide layer can be electrodeposited on the working electrode such that a Cu₂O film is formed that has a thickness of about 60 nm to about 120 nm. By way of example, the Cu₂O film can be electrodeposited using an electrolyte solution that includes 0.2 M cupric sulfate, 3 M of lactic acid and 3 M of sodium hydroxide with the pH value adjusted to 12. Although a pH value of 12 is used in this example, an electrolyte solution having pH values between about 8 to about 12 can be used in the electrodeposition of the Cu₂O film. Additionally, other concentration ranges for the chemicals of the electrolyte solution can be used as long as a substantially uniform Cu₂O film is formed on the working electrode during electrodepostion. The electrodeposition can be carried out at an elevated temperature, for example, about 40° C. Other deposition temperatures between room temperature and up to 60° C. can also be used. The deposition potential can be, for example, about −0.36 V versus Ag/AgCl reference electrode. The deposition potential, however, can be modified, such that it is higher or lower.

The sample combined with an alkaline solution that provides source of OH⁻ ions for reaction with glucose in the sample and Cu′ ions. The presence of OH⁻ ions and glucose can reduce Cu⁺ ions to Cu₂O. In some embodiments, the alkaline solution can include, for example, a 0.1M NaOH solution. Other chemicals, which can provide the OH— ions in the electrolyte can also be used.

The voltage source 22 can apply a voltage potential to the working electrode 14 and reference and/or counter electrode 16, 12, depending on the design of the sensor 10. The current between the working electrode 14 and counter electrode 16 can be measured with the measuring device or meter 24. Such current is dependent on interaction of glucose in the sample and OH⁻ ions with the Cu₂O on the working electrode.

The non-enzymatic glucose sensor can be used to detect glucose concentration in a sample using different electrochemical analytical techniques including, for example, chronoamperometry, amperometric measurement, and differential pulse voltammetry. The sample can include, for example, a bodily sample, such as (DPV) blood or serum.

The amount or level of current measured is proportional to the level or amount of glucose in the sample. In some embodiments, where the sample is blood, once the current level generated by the sample and electrolyte solution tested with the sensor is determined, the level can be compared to a predetermined value or control value to provide information for monitoring the presence or absence of glucose in the blood sample.

In other embodiments, where the sample is a bodily sample obtained from a subject, once the current level generated by the solution tested with the sensor is determined, the level can be compared to a predetermined value or control value to provide information for diagnosing or monitoring of the condition, pathology, or disorder in a subject that is associated with presence or absence of glucose, such as diabetes.

The current level generated by sample obtained from the subject can be compared to a current level of a sample previously obtained from the subject, such as prior to administration of a therapeutic. Accordingly, the methods described herein can be used to measure the efficacy of a therapeutic regimen for the treatment of a condition, pathology, or disorder associated with the level of the glucose in a subject by comparing the current level obtained before and after a therapeutic regimen. Additionally, the methods described herein can be used to measure the progression of a condition, pathology, or disorder associated with the presence or absence of glucose in a subject by comparing the current level in a bodily sample obtained over a given time period, such as days, weeks, months, or years.

The current level generated by a sample obtained from a subject may also be compared to a predetermined value or control value to provide information for determining the severity or aggressiveness of a condition, pathology, or disorder associated with glucose levels in the subject. A predetermined value or control value can be based upon the current level in comparable samples obtained from a healthy or normal subject or the general population or from a select population of control subjects.

The predetermined value can take a variety of forms. The predetermined value can be a single cut-off value, such as a median or mean. The predetermined value can be established based upon comparative groups such as where the current level in one defined group is double the current level in another defined group. The predetermined value can be a range, for example, where the general subject population is divided equally (or unequally) into groups, or into quadrants, the lowest quadrant being subjects with the lowest current level, the highest quadrant being individuals with the highest current level. In an exemplary embodiment, two cutoff values are selected to minimize the rate of false positive and negative results.

In some embodiments, different pulse voltammetry (DPV) can be used to measure the concentration of glucose in a sample using the Cu₂O layer formed as a non-enzymatic glucose sensor. For example, a sample comprising glucose can be combined with a 0.1 M NaOH electrolyte solution. The sample comprising glucose mixed with the NaOH electrolyte solution can then be placed onto the cuprous oxide covered working electrode element. A rest time interval (e.g., about 10 seconds) can be applied allowing the reaction between glucose and Cu₂O reaching a steady state. Other rest time intervals can also be used. In some embodiments, an applied voltage potential in the range of 0 to +0.75 V vs. the thick film printed Ag/AgCl reference electrode can be used in the DPV measurement of glucose. The detected current can then be compared to a control value to determine the concentration of glucose in the sample.

In other embodiments, chronoamperometry (CA) can be used to measure the concentration of glucose in a sample using the Cu₂O layer formed as a non-enzymatic glucose sensor. The electrochemical potential of the working electrode can be stepped and the resulting current from Faradaic process occurring at the working electrode (caused by the potential step) can then be monitored as a function of time. The measuring device for chronoamperometry can be simpler compared to the measuring device for DPV measurement, and the CA measurement can be considered as a verification of the effectiveness of DPV measurement. For example, CA measurement of glucose using Cu₂O formed layer can be carried out by applying an electrical potential window at a potential range of +0.35 V to +0.4 V vs Ag/AgCl reference electrode, and at an applied voltage of +0.35V with a step change to +0.4 V vs Ag/AgCl reference electrode to a selected glucose concentration solution using chronoamperometry (CA).

In still other embodiments, single-potential amperometric voltammetry can be used to measure the concentration of glucose in a sample using the Cu₂O layer formed as a non-enzymatic glucose sensor. Similar to chronoamperometry study, a sample comprising glucose and a NaOH solution can be added onto sensor surface for testing. A single potential of, for example, about +0.46 V vs Ag/AgCl reference electrode can then be applied. A rest time interval of, for example, about 10 s, can be used allowing the glucose and Cu₂O reaction to reach a steady state. The single-potential amperometric voltammetry responses on different glucose concentrations ranging can then be determined and compared to a control value solution.

The Example that follows illustrates embodiments of the present invention and are not limiting of the specification and claims in any way.

### Example

In this Example, Cu₂O was deposited by electrochemical deposition on a thin gold film sensor prototype. Glucose concentration ranges of 50-200 mg/dL in 0.1 M NaOH solution were detected using differential pulse voltammetry (DPV). Glucose detection was also performed in undiluted human serum using a minute quantity of 0.1 M NaOH, 3 μL serum containing glucose by DPV. Interference studies by uric acid and ascorbic acid showed that this Cu₂O based glucose sensor had good selectivity.

### Materials and Methods

### Apparatus and Reagents

Copper sulfate pentahydrate (#209198), lactic acid (#252476), sodium hydroxide (#306576), D-(+)-glucose (#G-8270), uric acid (#U-0881), L-ascorbic acid (#A5960) and human serum (#H3667) were obtained from Sigma Aldrich (St. Louis, Mo.). Sodium chloride (#S-271) was purchased from Thermo-Fisher (Pittsburgh, Pa.). Nickel chloride (#A0282977), and boric acid (#A0281874) were obtained from Acros Organics, Thermo-Fisher (Pittsburgh, Pa.). A CHI 660C Electrochemical Workstation (CH Instrument, Inc., Austin, Tex.,) was used for CV and DPV investigations. Other models of CHI 660 (Models A-E) could also be used. All experiments were conducted at room temperature. X-ray photoelectron spectroscopy (XPS) was performed by a PHI (Physical Electronics Inc., Chanhassen, Minn.) Versaprobe 5000 Scanning X-Ray Photoelectron Spectrometer using an Aluminum Kα X-ray radiation (50 W, 15 KV, 1486.6 eV, 100 μm spot size on the sample) served as the excitation source. The analyzer was operated at a constant pass energy of 23.5 eV. Under these conditions, the Au 4f₇₁₂ photoelectron peak was recorded with 0.7 eV at a binding energy of 84.0 eV. The calibration of the binding energy of the spectra was performed with the C is peak of the adventitious carbons, which was at 284.8 eV. The spectra of each sample was obtained with a short acquisition time of 10 minutes to examine C 1 s, Cu 2p and Cu LMM XPS regions in order to avoid, as much as possible, the photo-reduction of species. The information from the outermost ˜10 nm of the surface was converted to a depth profile using data acquired in an angular dependent XPS experiment. When the angle between sample normal and the analyzer entrance was increased, with the X-ray source and analyzer kept in fixed positions, the photoelectrons originated from an increasingly surface localized zone. Spectrums were acquired at take-off angles of 0°, 45° and 80° in order to obtain information about the composition as a function of the depth.

### Fabrication of Non-Enzymatic Cu₂O Glucose Sensor

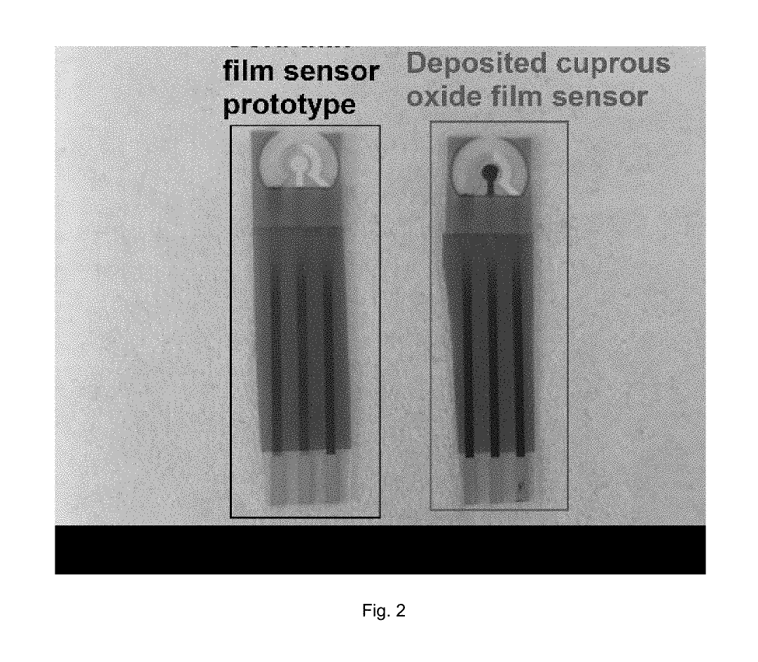

Sputtered thin gold film sensor prototype used in this example had been described in U.S. patent application Ser. No. 09/466,865, which is incorporated by reference in its entirety. Electrochemical deposition of the cuprous oxide film on the thin gold film working sensor element was carried out. A mixture of 0.2 M cupric sulfate, 3 M of lactic acid and approximately 3 M of sodium hydroxide for adjusting pH value to 12 was used as electrolyte in this Cu₂O deposition. A water bath was used for maintaining the electrolyte solution at 40° C. 20 μL of prepared electrolyte solution was casted on the sensor and linear sweep voltammetry was applied for deposition of cuprous oxide. Linear sweep voltammetry of potential from −0.8 V to −0.1 V was applied for deposition. Electrochemical deposition potential was at −0.36 V versus the thick-film printed Ag/AgCl reference electrode. The darker color of the working electrode (in the center of the sensor prototype) shown in FIG. 2 shows the deposition of the cuprous oxide layer on the thin gold film working electrode. After deposition, the cuprous oxide thin film sensor was washed with DI water, dried by nitrogen and ready for use.

### Results

Surface Characterization of Cuprous Oxide Layer with XPS

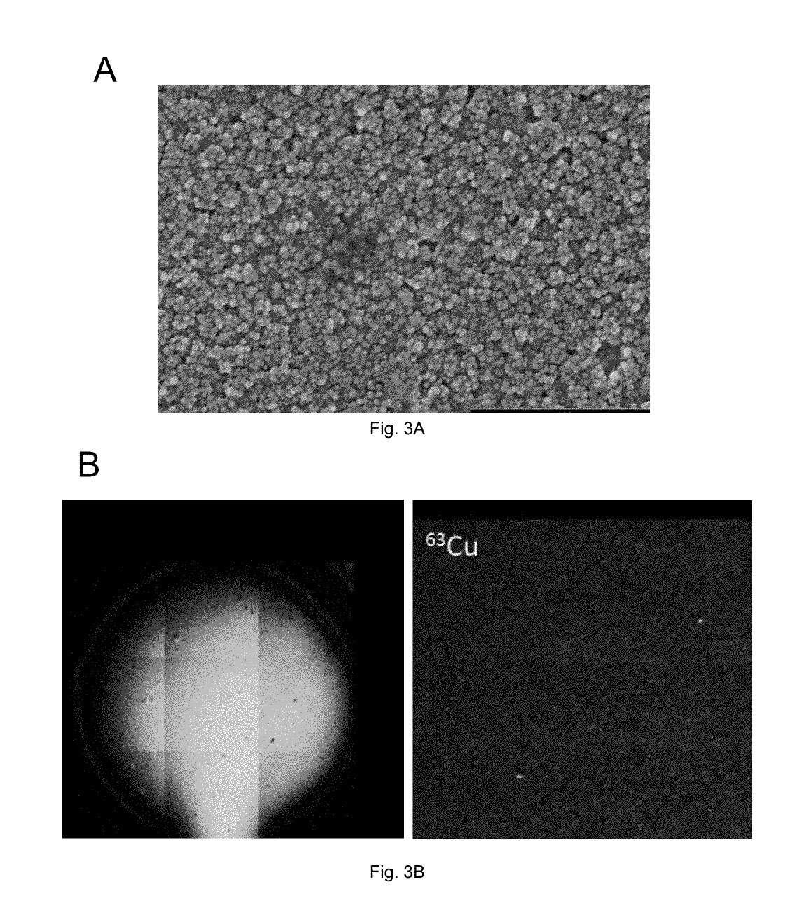

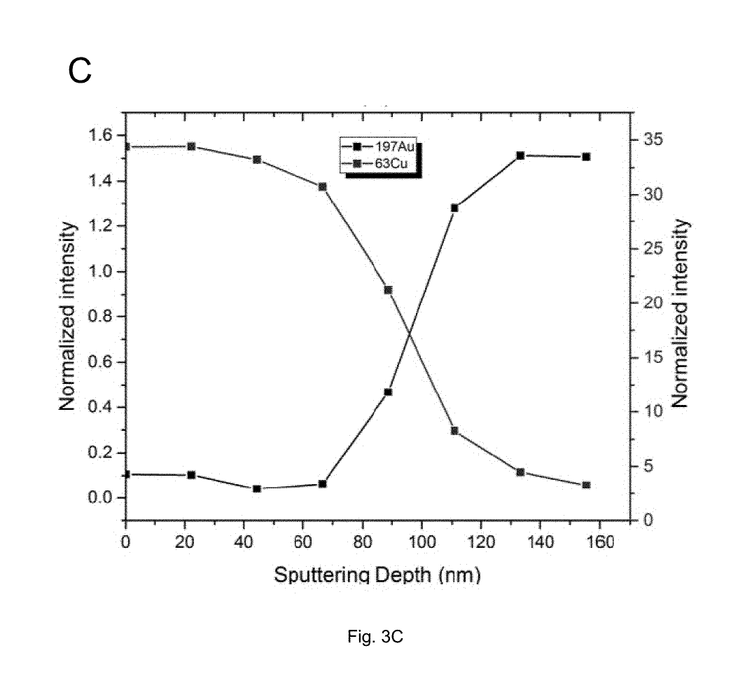

X-ray photoelectron spectroscopy (XPS) was used for examining the formation of cuprous oxide film on the thin gold film working electrode. The chemical shift of the Cu 2p3/2 photoelectron peak was not detectable with the energy resolution of the XPS. The presence and the intensity of the satellite peaks for the kinetic energy was at 940-945 eV region, indicating a binding energy of 570 eV. The peak shape and position of Cu LMM were experimentally verified and matched with the reference data. The experimental data from 2 different samples along with the reference data are shown in FIG. 3. The decreasing of the take-off angle increased the height of the Cu2p due to the smaller of the take-off angle resulting in the sample became closer to the detector and with more photoelectrons reaching the detector. Consequently, the results also confirmed the presence of Cu₂O by forming weak satellites in between Cu 2p3/2 and Cu 2p1/2 peaks. This conclusion was supported from the spectra acquired at various take-off angles proving that a uniform Cu₂O layer was formed.

### Electrochemical Measurement of Glucose by Differential Pulse Voltammetry (DPV)

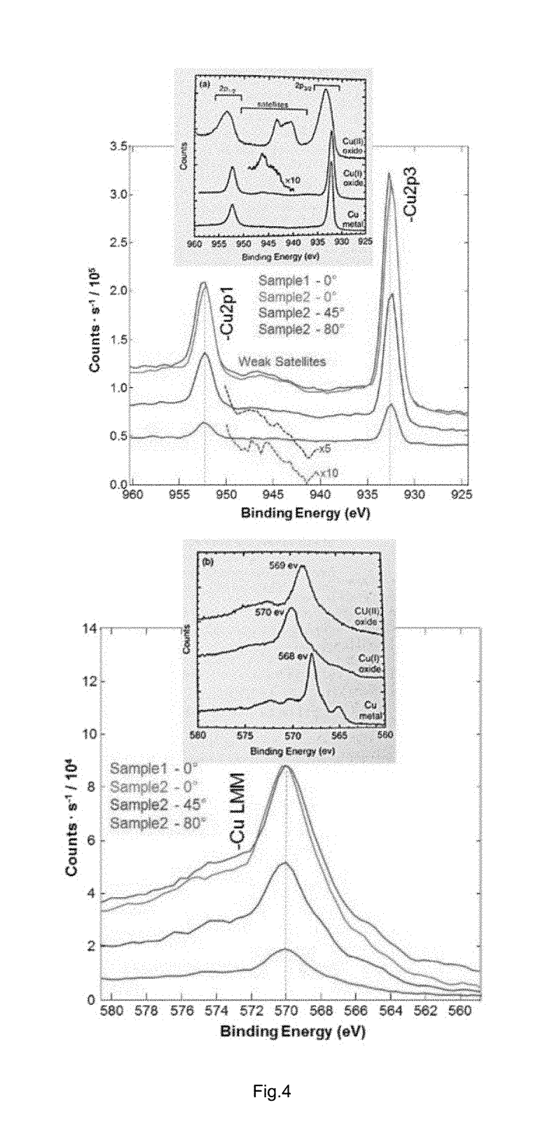

Glucose was prepared in a 0.1 M NaOH solution with the concentration ranging from 50 mg/dL to 200 mg/dL. Based on the mechanism of reaction of glucose with cuprous oxide in alkaline solution, an increase of the anodic peak current at a potential of approximately +0.4 V versus a thick-film printed Ag/AgCl reference electrode was observed with increasing concentration of glucose. In a typical experimental run, 20 μL of 0.1 M NaOH with known glucose solution was drop casted on the cuprous oxide based sensor. A rest time was set for 10 s allowing the hydroxide ion to first oxidize the cuprous oxide. DPV was then conducted in the range of 0 to +0.75 V versus the thick-film printed Ag/AgCl reference electrode. DPV measurements of different glucose concentrations in 0.1 M NaOH solution are shown in FIG. 4A. The calibration curve based on the DPV current output and concentration of glucose is shown in FIG. 4B. A linear relationship Y=0.024X+1.46 with adjusted R square value of 0.978 (n>5) is established demonstrating excellent sensitivity and reproducibility of cuprous oxide film based glucose sensor.

### Glucose Detection by Chronoamperometry (CA) and Single-Potential Amperometric Voltammetry

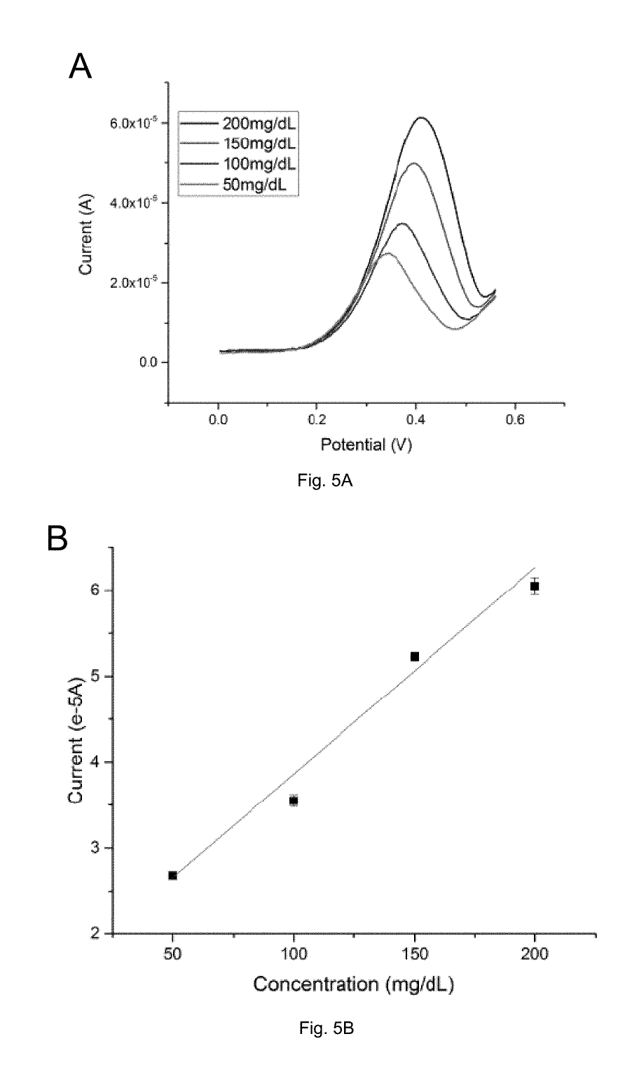

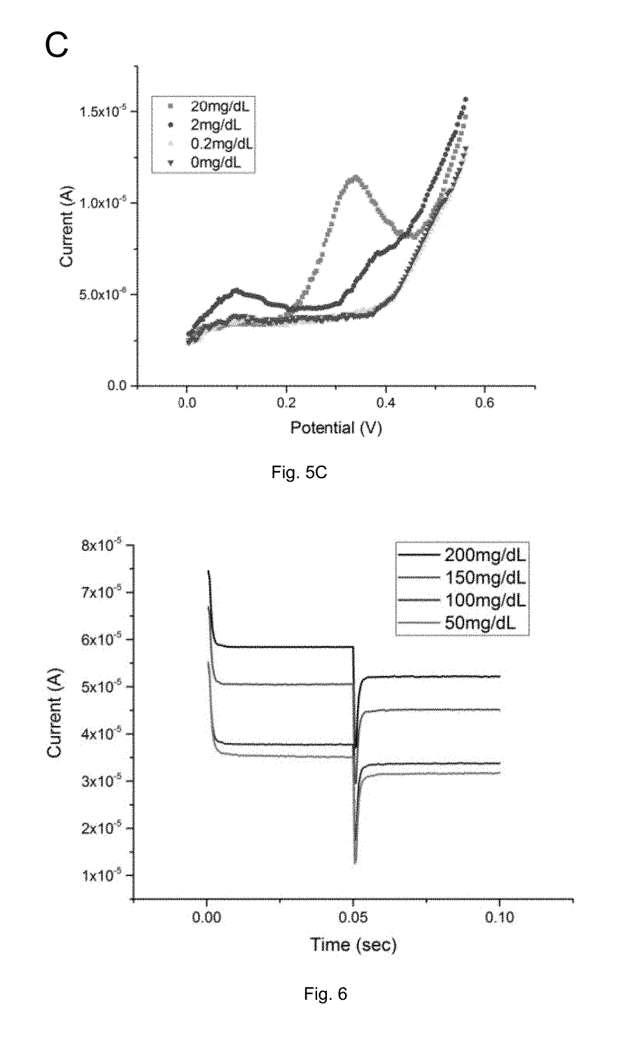

Determination of glucose using a Cu₂O thin layer based sensor and DPV measurement demonstrated that the detection technique was very effective. However considering the electrochemical complexity of DPV compared to commonly used CA and single-potential amperometric voltammetry, glucose detection by CA and single-potential amperometric voltammetry were also carried out. The results validated that Cu₂O thin layer based sensor for glucose detection with different electrochemical detection techniques in addition to DPV measurement was successful. Furthermore, the results of this study verified the effectiveness of a Cu₂O thin layer based sensor for glucose detection in an alkaline medium. FIG. 5 shows the chronoamperometry (CA) response of a Cu₂O thin layer based sensor to different glucose concentration in 0.1M NaOH test medium. In this CA measurement, a voltage of +0.35 V versus Ag/AgCl reference was applied and then with a step change of potential to +0.4 V in voltage.

FIG. 6 shows the single-potential amperometric voltammetry responses on glucose concentrations ranging from 50 mg/dL to 200 mg/dL in 0.1 M NaOH solution. There was no potential step change in single-potential amperometric voltammetry, compared to CA measurement. Thus, this cuprous oxide film based sensor also showed an excellent response in the single-potential amperometric voltammetry at a single potential of +0.5 V versus Ag/AgCl reference electrode. A rest time of 10 s was used allowing the reaction between glucose and Cu₂O to reach a steady state. The single-potential amperometric voltammetry took 0.3 s to complete.

The detection responses of both chronoamperometry and single-potential amperometric voltammetry as demonstrated in FIGS. 5 and 6 provided verification of the detection of glucose of a Cu₂O thin layer based sensor in an alkaline medium by DPV technique.

### Detection of Glucose in Undiluted Human Serum by DPV

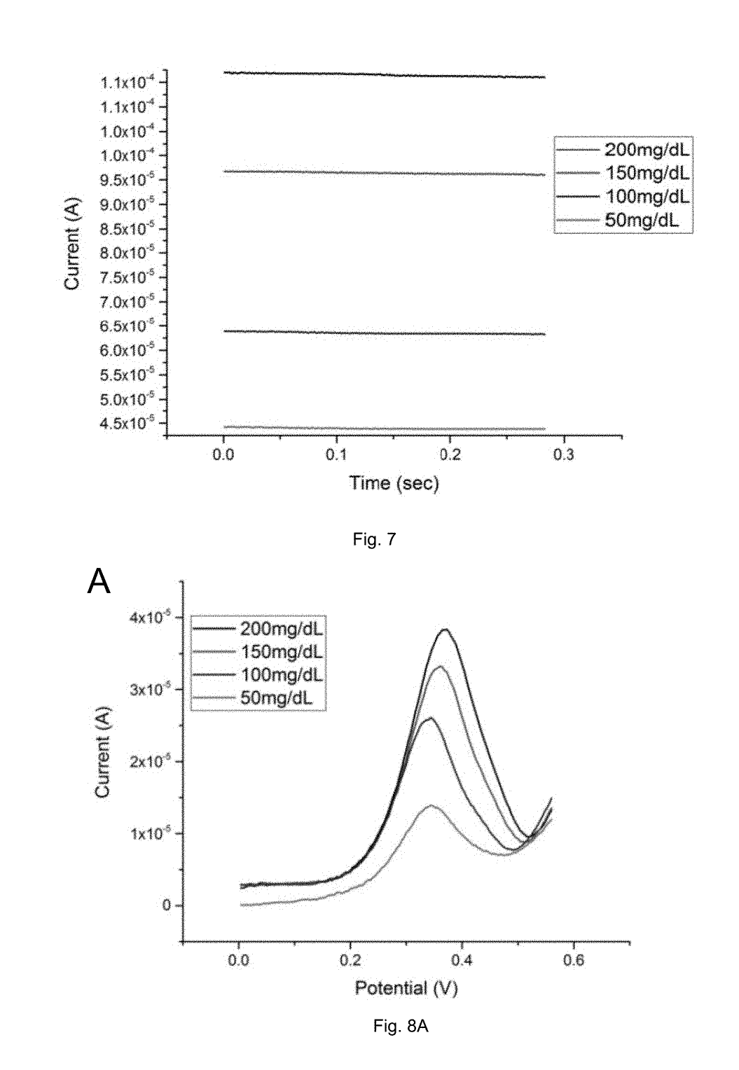

In a typical run, 3 μL of glucose in serum solution was mixed with 3 μL of 0.1M NaOH solution. Then, this 6 μL of the mixed solution was placed on the sensor and DPV was applied as described in section 3.2. FIG. 7A shows the DPV detection responses of the sensor for glucose solutions in human serum ranging from 50 mg/dL to 200 mg/dL. FIG. 7B shows the calibrated linear fit for the DPV results with an equation of Y=0.016X+0.847 and adjusted R square value of 0.929 (n>5).

DPV measurements demonstrated that this Cu₂O thin layer based glucose sensor could be used effectively in blood serum by adding a minor volume of 3 μL of 0.1 M NaOH to the glucose test sample. This 3 μL of 0.1 M NaOH or similar hydroxide ion containing solutions could be applied to the glucose test sample in serum with minimum inconvenience.

### Interference Study of Cu₂O Thin Layer Based Sensor for Glucose Detection

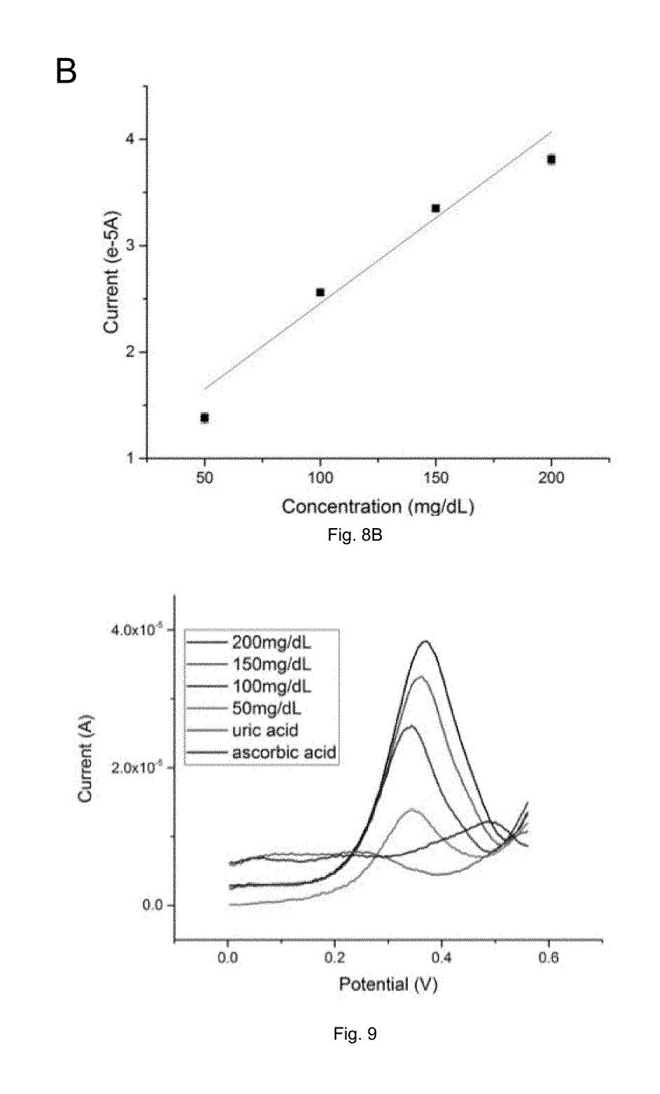

Interference testing was important to ensure the selectivity of the Cu₂O thin layer based sensor for glucose detection. Two common inference chemicals of glucose sensing, ascorbic acid and uric acid, were used in this example. The actual quantities of these interfering species are relatively minute compared with the quantity of glucose in human blood. However, a relatively large quantity of ascorbic acid or uric acid was used in this interference study demonstrating that the selectivity of this Cu₂O thin layer based glucose sensor was excellent. 100 mg/dL of ascorbic acid and 100 mg/dL of uric acid were prepared individually in undiluted human serum. Both ascorbic acid and uric acid at this high concentration level did not contribute any current in the DPV measurement of glucose detection as shown FIG. 8. The results demonstrated that the selectivity of this Cu₂O thin layer based sensor for glucose sensing in an alkaline medium was excellent.

### Non-Enzymatic Metallic Catalyst Based Glucose Sensors in Alkaline Solution

In additional to cuprous oxide thin film, metallic catalysts, such as nickel, platinum, and copper, also showed promising ability in reaction with glucose in an alkaline condition. The reaction mechanism demonstrated that the alkaline solution oxidized metal catalysts, then glucose reduced the oxidized metal producing gluconic acid. Thus, the performance of metallic catalysts, including nickel, platinum and copper for detection of glucose in alkaline solution, were examined and compared with the cuprous oxide thin film sensor.

### Electrochemical Deposited Copper Film for the Detection of Glucose in Alkaline Solution

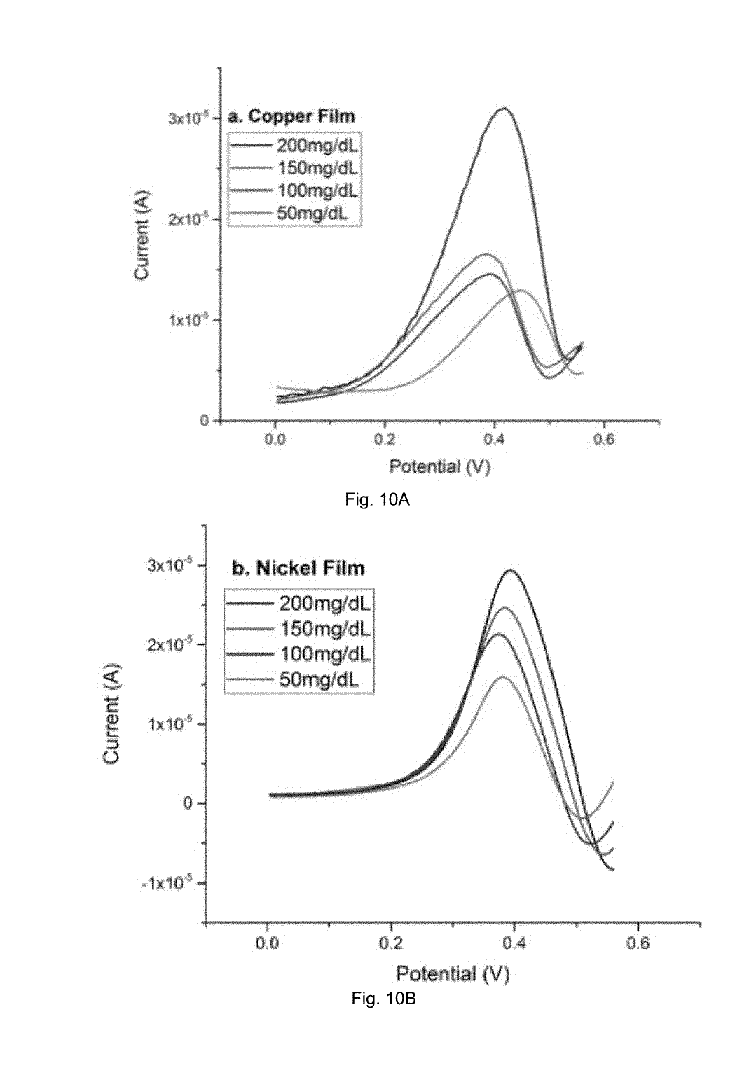

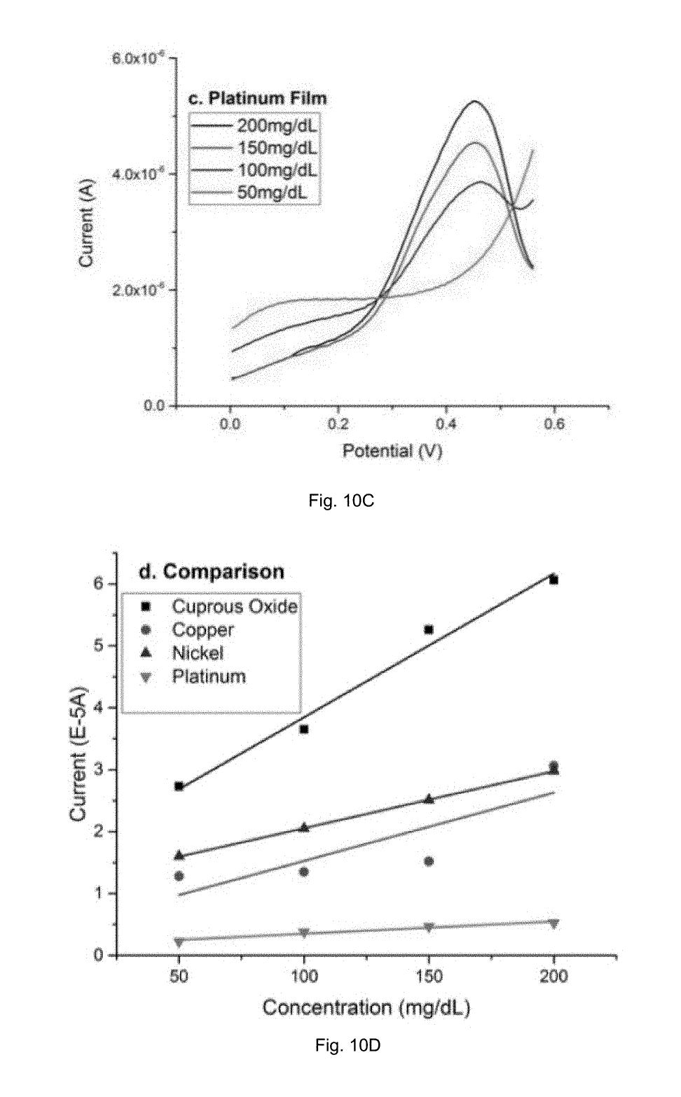

Copper was used for the detection of organic compounds based on its oxidation activity in alkaline solution, including using cuprous and cupric oxides as discussed above. In order to assess the effect and the role of copper serving as a metal based sensor for glucose detection, copper was electrochemically deposited onto the thin gold film sensor prototype and evaluated. Cathodic reduction of Cu⁺² ions from an electrolyte was employed for the electrochemical deposition of copper onto the thin gold film sensor prototype. Typically, an electrolyte of 0.05M of CuSO₄ and 0.1M of H₂SO₄ in aqueous solution was used for the copper deposition. 20 μL of the electrolyte was placed on the gold thin film sensor and linear sweep voltammetry of −0.9 V to −0.3 V was applied for deposition of copper at room temperature. Cathodic peak current at −0.86 V versus Ag/AgCl reference electrode was observed for this reduction reaction. DPV was applied, and the oxidation reaction between glucose and copper took place at around +0.40 V vs. Ag/AgCl as reference electrode. FIG. 9A shows the anodic currents of the DPV measurements of glucose concentrations of 50-200 mg/dL in a 0.i M NaOH solution. The anodic peak currents appeared at approximately +0.40 V versus the thick-film printed Ag/AgCl reference electrode.

### Electrochemical Deposited Nickel Film for Detection of Glucose in Basic Solution

Nickel was a good biological reaction catalyst with a significant chemical activity. Nickel was an active material for glucose detection in the presence of OH. The performance of nickel on the detection of glucose was evaluated and compared to the cuprous oxide thin film layer based sensor for glucose detection. Nickel was deposited electrochemically onto the thin gold film sensor prototype. An electrolyte containing 0.14 M NiCl₂, 1M NaCl, 0.5M H₃BO₃ and a copious amount of HCl for adjusting the solution pH value to around 1.5 was prepared for the deposition of nickel. 20 μL of an electrolyte was placed on the gold thin film sensor and linear sweep voltammetry of −1.2 V to −0.7 V was applied for deposition of nickel at room temperature. An increasing reduction cathodic deposition peak was observed at −1 V vs. Ag/AgCl as reference electrode. Differential pulse voltammetry was applied and an anodic peak was obtained at +0.38V versus the thick film printed Ag/AgCl reference electrode. FIG. 9B shows the differential pulse voltammetry graph for the detection of glucose in 0.1 M NaOH solution using the electrochemically deposited nickel thin film sensor covering the glucose concentration range of 50 mg/dL to 200 mg/dL.

### Sputtered Thin Platinum Film Sensor for the Detection of Glucose in Alkaline Solution

Platinum is well-accepted as a bioactive metal or catalyst for the detection of organic compounds, including carbohydrates, amino acids and glucose. Therefore, the detection of glucose using a platinum based sensor was also studied in this research endeavor. The fabrication of the thin platinum film sensor prototype was identical to the process of the thin gold film sensor prototype with the only difference that platinum (50 nm thickness) was used for the working and the counter electrodes stead of gold. Sputtering physical vapor deposition, laser ablation and thick-film printing technologies were used, and the platinum thin film sensor could also be fabricated by roll-to-roll cost-effective manufacturing process. The testing protocol of glucose using this platinum thin film based sensor was identical to the testing procedure described in section 3.2. DPV was applied and anodic peak currents for different glucose concentrations were observed at approximately +0.43 V versus the thick-film printed Ag/AgCl reference electrode. FIG. 9C shows the DPV measurements of various glucose concentrations of glucose in 0.1 M NaOH solution ranging from 50 mg/dL to 200 mg/dL.

FIG. 9D summarized the performance of these non-enzymatic glucose sensors by displaying their calibration curves based on the data acquired. Compared with metal films sensor for detection of glucose in alkaline solution, cuprous oxide demonstrated the highest current outputs, showing a promising application as a non-enzymatic glucose sensor.

A non-enzymatic cuprous oxide (Cu₂O) thin layer based sensor for the detection of glucose in an alkaline medium, 0.1 NaOH solution over the glucose concentration range of 50-200 mg/dL was successfully developed using differential pulse voltammetry (DPV) measurement. X-ray photoelectron spectroscopy (XPS) confirmed the formation of cuprous oxide, Cu₂O layer on the thin gold film sensor prototype. The evaluation of glucose in both phosphate-buffered saline (PBS) and undiluted human serum were carried out. The 0.1 M NaOH alkaline solution used was minute, 3 μL in a total of 6 μL test medium. Neither ascorbic acid nor uric acid, even at a high concentration level of 100 mg/dL in serum, interfered with the cuprous oxide (Cu₂O) thin layer based sensor in glucose measurement, demonstrating the selectivity of this non-enzymatic cuprous oxide (Cu₂O) thin layer based sensor was excellent. Chronoamperometry (CA) and single-potential amperometric voltammetry were also used in the glucose detection experimentally using this cuprous oxide (Cu₂O) thin layer based sensor. The positive results verified the validity of detecting glucose in a 0.1 M NaOH alkaline medium by DPV measurement. Nickel, platinum and copper were commonly used metals for non-enzymatic glucose detection. The performance of these metal based sensors for glucose detection using DPV technique were experimentally evaluated. Cuprous oxide (Cu₂O) thin layer based sensor showed the best sensitivity for glucose detection among the sensors evaluated.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

Having described the invention, we claim:

1. A non-enzymatic sensor for the detection of glucose comprising:
a substrate;
a working electrode formed on a surface of the substrate;
a counter electrode formed on the surface of the substrate;
a dielectric layer covering a portion of the working electrode and counter electrode and defining an aperture exposing other portions of the working electrode and counter electrode; and
a cuprous oxide (Cu₂O) film electrodeposited on the working electrode.

2. The sensor of claim 1, wherein the working electrode and the counter electrode comprise metalized films.

3. The sensor of claim 1, wherein the working electrode and counter electrode independently comprise gold, platinum, palladium, silver, carbon, alloys thereof, and composites thereof.

4. The sensor of claim 1, the metalized films are provided on the surface of the substrate by sputtering or coating the films on the surface and wherein the working electrode and the counter electrode are formed using laser ablation

5. The sensor of claim 1, further comprising a reference electrode on the surface of the substrate, the dielectric covering a portion of the reference electrode.

6. The sensor of claim 1, wherein the working electrode and the counter electrode comprise gold films, and the reference electrode comprises an Ag/AgCl film.

7. The sensor of claim 1, further comprising a measuring device for applying voltage potentials to the working electrode and counter electrode and measuring the current flow between the working electrode and counter electrode.

8. The sensor of claim 1, wherein the Cu₂O film has a thickness of about 60 nm to about 120 nm.

9. A method of detecting glucose in a sample, the method comprising:
providing a non-enzymatic sensor that includes a substrate; a working electrode formed on a surface of the substrate; a counter electrode formed on the surface of the substrate; a dielectric layer covering a portion of the working electrode and counter electrode and defining an aperture exposing other portions of the working electrode and counter electrode; and a cuprous oxide (Cu₂O) film electrodeposited on the working electrode;
combining the sample with an alkaline solution;
applying a volume of the sample and alkaline solution to the working electrode;
applying voltage potentials to the working electrode and counter electrode;
measuring the current flow between the working electrode and counter electrode; and
comparing the measure current flow to a control value to determine the concentration of glucose in the sample.

10. The method of claim 9, wherein the working electrode and the counter electrode comprise metalized films.

11. The method of claim 9, wherein the working electrode and counter electrode independently comprise gold, platinum, palladium, silver, carbon, alloys thereof, and composites thereof.

12. The method of claim 9, the metalized films are provided on the surface of the substrate by sputtering or coating the films on the surface and wherein the working electrode and the counter electrode are formed using laser ablation

13. The method of claim 9, further comprising a reference electrode on the surface of the substrate, the dielectric covering a portion of the reference electrode.

14. The method of claim 9, wherein the working electrode and the counter electrode comprise gold films, and the reference electrode comprises an Ag/AgCl film.

15. The method of claim 9, further comprising a measuring device for applying voltage potentials to the working electrode and counter electrode and measuring the current flow between the working electrode and counter electrode.

16. The method of claim 9, wherein the Cu₂O film has a thickness of about 60 nm to about 120 nm.

17. The method of claim 9, wherein the sample comprises blood or serum.

18. The method of claim 9, wherein the alkaline solution comprises a NaOH solution.
